(19) United States
(12) Patent Application Publication
(10) Pub. No.: US 2012/0202214 A1
(43) Pub. Date: Aug. 9, 2012

(54) METHOD FOR DETECTING FUSION GENE

(76) Inventor: Kazuya Omi, Chuo-ku (JP)

(21) Appl. No.: 13/500,497

(22) PCT Filed: Sep. 28, 2010
(86) PCT No.: PCT/JP2010/066768

§ 371 Date: Apr. 5, 2012

(30) Foreign Application Priority Data
Oct. 6, 2009 (JP) 2009-232473

(51) Int. Cl. C12Q 1/68; G01N 33/50; G01N 27/447; C07H 21/04; G01N 21/64

(52) U.S. Cl. 435/6.12; 536/24.33; 204/450

(57) ABSTRACT

Disclosed is a means which enables simple and rapid detection of the presence of any fusion genes including even unknown fusion genes. The method for measuring a fusion gene(s) according to the present invention is applied to a sample separated from a living body, and comprises: measuring expressions of a 5′-region and a 3′-region of one of the component genes of the fusion gene(s) which may be present in the living body, wherein said 5′-region is upstream of and said 3′-region is downstream of a fusion point in said one of the component genes; and comparing the expression of the 5′-region with the expression of the 3′-region. The fusion gene to be measured is e.g. a fusion gene between ALK gene and another gene.

### TECHNICAL FIELD

The present invention relates to a method for measuring a fusion gene(s) which may be present in a living body.

### BACKGROUND ART

It is known that fusion genes may cause cancer in some cancer patients. For example, anaplastic lymphoma kinase (ALK) gene, one of the receptor tyrosine kinases, is known to become oncogenic by fusion with various genes (Non-patent Document 5).

EML4-ALK fusion gene is a gene generated by fusion between a portion of 5′-region of Echinoderm Microtubule-associated protein-Like 4 (EML4) gene and 3′-region of ALK gene, and is known to cause some non-small-cell lung cancers (Patent Document 1, Non-patent Document 1). As a method for detection of EML4-ALK fusion gene, a method in which EML4-ALK fusion gene mRNA is amplified by RT-PCR has been reported (Non-patent Document 2).

It is known that EML4-ALK fusion gene includes at least 9 variants whose fusion points are different from one another (Non-patent Document 3). Therefore, if it is desired to detect the expression of all of the fusion genes efficiently and simply by using the known method, complex multiplexing is required for modifying the known method into a system in which many primers and probes are used. A lot of costs and time are needed for such multiplexing, which is problematic. In addition, besides EML4 gene, a plurality of genes have been reported as a fusion ALK gene (Non-patent Document 4, Non-patent Document 5), and therefore it is highly likely that new fusion genes will be found. It is impossible to detect unknown fusion genes whose sequences are not yet identified by the known method in which specific primers and/or probes are designed for individual fusion genes.

### PRIOR ART DOCUMENTS

### Patent Documents

Patent Document 1: JP 2008-295444 A

### Non-Patent Documents

Non-patent Document 1: Nature 2007; 448:561-566
Non-patent Document 2: Clin. Can. Res. 2008; 14:6618-6624
Non-patent Document 3: J. Clin. Oncol. 2009; 27:1-4
Non-patent Document 4: Clin. Can. Res. 2009; 15:3143-3149
Non-patent Document 5: Biochem. J. 2009; 420:345-361

### SUMMARY OF THE INVENTION

### Problems to be Solved by the Invention

Accordingly, an object of the present invention is to provide a simple detection system for fusion genes including those not yet known.

### Means for Solving the Problems

The present inventor has intensively studied to find that the nature of the EML4-ALK fusion gene is “X (an arbitrary gene)+3′-region (comprising kinase domain) of ALK gene”, and that the presence of any fusion genes can be easily and rapidly detected regardless of the kind of their fusion partners by measuring expression level of regions upstream and downstream of the fusion point in ALK gene. Furthermore, he has examined more than 50 kinds of primer sets to find combinations of the primers which can attain simultaneous amplification including internal control while reducing non-specific amplification, thereby completing the present invention.

That is, the present invention provides a method for measuring a fusion gene(s), which is applied to a sample separated from a living body, said method comprising: measuring expressions of a 5′-region and a 3′-region of one of the component genes of the fusion gene(s) which may be present in the living body, wherein said 5′-region is upstream of and said 3′-region is downstream of a fusion point in said one of the component genes; and comparing the expression of the 5′-region with the expression of the 3′-region. The present invention also provides a reagent for detection of a fusion gene(s) formed by fusion with ALK gene, said reagent comprising a 5′-region detection primer set composed of the base sequences shown in SEQ ID NOs: 3 and 4 of the SEQUENCE LISTING, respectively, and a 3′-region detection primer set composed of the base sequences shown in SEQ ID NOs: 5 and 6, respectively. The present invention further provides a reagent for detection of a fusion gene(s) formed by fusion with ALK gene, said reagent comprising a 5′-region detection primer set composed of the base sequences shown in SEQ ID NOs: 3 and 4 of the SEQUENCE LISTING, respectively, and a 3′-region detection primer set composed of the base sequences shown in SEQ ID NOs: 20 and 21, respectively.

### Effects of the Invention

By the present invention, a novel method which can comprehensively detect fusion genes which may be present in living bodies regardless of the kind of the fusion partner was provided. According to the present invention, the expression levels of just two regions of a gene are measured, so that any fusion genes generated by fusion with any fusion partner can be detected, thereby greatly reducing time and costs spent on detection of various kinds of fusion genes. For example, as an ALK fusion gene, those generated by fusion between an ALK 3′-region comprising the kinase domain of ALK gene and a fusion partner such as EML4 gene or the like are known. The method of the present invention can detect any fusion genes in which an upstream region of ALK gene is lost, and therefore can detect even novel fusion genes which have not been specifically identified yet, which is one of the great advantages of the present invention. Particularly, by using the specific primer sets the present inventor has completed, PCR can be carried out in a single tube, including an internal control. Therefore, the present invention is very advantageous as a simple and rapid detection system for ALK fusion genes. The presence of fusion genes such as EML4-ALK fusion gene and the like has been confirmed in some of various cancer patients, and inhibitors which inhibit the activity of fusion gene products may be effective for cancer treatment in such fusion gene-expressing patients. The patients who should be treated with inhibitors can be selected from cancer patients by determining whether a fusion gene(s) is(are) expressed in cells, tissues, or body fluids derived from the cancer patients in accordance with the method of the present invention. Therefore, the method of the present invention is especially useful in the field of cancer therapy.

### BRIEF DESCRIPTION OF THE DRAWINGS

FIG. 1 shows the results of the measurement of the 5′-region and the 3′-region of ALK gene on RNA samples extracted from H2228 cells (EML4-ALK fusion gene positive, ALK gene negative), A549 cells (EML4-ALK fusion gene negative, ALK gene negative) and SK-N-DZ cells (EML4-ALK fusion gene negative, ALK gene positive) in Example 1.

FIG. 2 shows the results of the electrophoresis of the amplification products obtained by simultaneous amplification of the 5′-region and the 3′-region of ALK gene and the internal control gene TBP from RNA samples extracted from H2228 cells, A549 cells and SK-N-DZ cells in Example 2.

FIG. 3 shows the results of the measurement of the amplification products obtained by simultaneous amplification of the 5′-region and the 3′-region of ALK gene and the internal control gene TBP from RNA samples extracted from H2228 cells, A549 cells and SK-N-DZ cells using fluorescent probes in Example 3.

### MODE FOR CARRYING OUT THE INVENTION

The fusion gene(s) to be measured in the present invention is(are) a gene generated by fusion between a part of a certain gene and a part of another gene as a result of a translocation (including inversion) within a chromosome or between chromosomes. It is known that as a result of a translocation a gene which is not expressed in a certain tissue under normal circumstances may be fused to a promoter region of another gene which can be expressed in the said certain tissue, thereby being expressed in the said certain tissue to cause various diseases including cancer. Most of the known fusion genes have been identified as an abnormal gene involved in various diseases. Also in the case of the fusion gene(s) to be measured in the present invention, one of the component genes of the fusion gene(s) is usually a gene, the whole of which is not naturally expressed in a normal tissue but a portion of which is allowed to be expressed in the said tissue by gene fusion, and the 5′-region and the 3′-region of the said one of the component genes is measured. However, the requirements of the fusion gene(s) are not restricted by the descriptions above, since whether and how much a fusion gene(s) is(are) expressed can be determined based on the ratio of the expression levels of the 5′-region and the 3′-region as described hereinafter even if the full-length of the component gene is expressed in a normal tissue.

Preferred examples of the fusion gene include those formed by fusion between a gene such as ALK gene, ABL gene, retinoic acid receptor gene, SSX gene, AML1 gene or the like and another gene. For example, ALK gene is a gene encoding receptor tyrosine kinase. Various fusion genes formed by fusion between the 3′-region of ALK comprising its kinase domain and the 5′-region of another gene are known, and they are known to be oncogenic (see, e.g. Non-patent Document 5). However, the scope of the present invention is not restricted to these specific examples, and not only fusion genes involved in various diseases such as cancer but also various naturally-occurring fusion genes not involved in any disease are included therein.

ALK gene (SEQ ID NO: 1, GenBank NM_{—}004304) is located on human chromosome 2 and consists of 29 exons. The kinase domain spans 4256 nt to 5083 nt (i.e. the 4256th to the 5083rd bases). The exon regions of ALK gene shown in SEQ ID NO: 1 are shown in Table 1 below. As a fusion gene between ALK gene and another arbitrary gene (this fusion gene is hereinafter also referred to as “ALK fusion gene” for short), fusion genes between ALK and various fusion partners are known, as described in Non-patent Document 5. Examples of the known ALK fusion gene are shown in SEQ ID NOs: 9 to 18 in SEQUENCE LISTING. SEQ ID NOs: 9 to 17 are the base sequences of EML4-ALK fusion genes, and SEQ ID NO: 18 is the base sequence of KIF5B-ALK fusion gene. These ALK fusion genes are generated by fusion between the region of exons 20 to 29 of ALK gene in which the kinase domain is included and a 5′-region of another gene in which promoter region is included, and their expression is observed in some non-small-cell lung cancer patients. As an ALK fusion gene, those in which ALK is fused to another gene at the 5′-end of exon 20 (SEQ ID NOs: 9 to 12, 14, 18), those in which ALK is fused together with several tens of by of the neighboring intron region (SEQ ID NOs: 15, 16), and those in which several tens to a hundred and several tens of by of the 5′-end region of exon 20 are lost (SEQ ID NOs: 13, 17) are known. As for the genes listed above as preferred examples besides ALK gene, their sequences are known, being registered in databases such as GenBank, and examples of their fusion genes are also known. Therefore, those skilled in the art can carry out the method of the present invention on such fusion genes, in the same manner as on ALK fusion genes.

TABLE 1

| | location (nt) |
|---|---|
| exon 1 | 1-1574 |
| exon 2 | 1575-1694 |
| exon 3 | 1695-1859 |
| exon 4 | 1860-2061 |
| exon 5 | 2062-2189 |
| exon 6 | 2190-2321 |
| exon 7 | 2322-2453 |
| exon 8 | 2454-2554 |
| exon 9 | 2555-2724 |
| exon 10 | 2725-2819 |
| exon 11 | 2820-2948 |
| exon 12 | 2949-3111 |
| exon 13 | 3112-3262 |
| exon 14 | 3263-3394 |
| exon 15 | 3395-3539 |
| exon 16 | 3540-3722 |
| exon 17 | 3723-3821 |
| exon 18 | 3822-3974 |
| exon 19 | 3975-4079 |
| exon 20 | 4080-4266 |
| exon 21 | 4267-4357 |
| exon 22 | 4358-4422 |
| exon 23 | 4423-4552 |
| exon 24 | 4553-4650 |
| exon 25 | 4651-4743 |
| exon 26 | 4744-4845 |
| exon 27 | 4846-4980 |
| exon 28 | 4981-5071 |
| exon 29 | 5072-6220 |

In the method of the present invention, one of the component genes which constitute fusion genes is focused on, and the expressions of the two region, i.e., a 5′-region of the gene which is upstream of the fusion point and a 3′-region of the gene which is downstream of the fusion point, are measured. In the present specification, one of the component genes whose 5′-region and 3′-region are to be measured may also be referred to as “target gene” for convenience. The term “measurement” includes detection, quantification and semi-quantification.

As used herein, “fusion point” means the boundary between the region lost by fusion and the region contained in a fusion gene. Focusing on a fusion gene, “fusion point” may also be understood as the boundary at which two different genes are fused. For example, in the case of ALK fusion genes shown in SEQ ID NOs: 9 to 12, 14 and 18, the 5′-end of exon 20 of ALK gene (i.e. 4080 nt in SEQ ID NO: 1) is the fusion point.

The 5′-region and the 3′-region to be measured may be selected from a region upstream of and a region downstream of the fusion point, respectively. It should be understood that, as exemplified by known ALK fusion genes, the fusion point is not necessarily restricted to the just one point in a certain component gene, and that the position of the fusion point may be different among fusion gene variants composed of the same combination of component genes by several tens of bases or more. Therefore, it is desirable to select the 5′-region to be measured from a region upstream of the known fusion point located nearest to the 5′-end. Similarly, it is desirable to select the 3′-region to be measured from a region downstream of the known fusion point located nearest to the 3′-end. Furthermore, in view of covering unknown, not specifically-identified variants, it is preferred to select regions to be measured from a region at a sufficient distance of, e.g., about 200 bases, preferably 500 bases or more, from the most upstream fusion point or the most downstream fusion point.

For example, as described above, most of the known ALK fusion genes are those in which exon 20 and its downstream region of ALK gene are fused to a fusion partner. Therefore, when measuring the ALK fusion gene(s), the 5′-region may be selected from a region of ALK exon 19 and its upstream region, and the 3′-region may be selected from a region of ALK exon 20 and its downstream region. In addition, because an ALK fusion gene in which up to 49 by in the 5′-end region of exon 20 are lost is known (SEQ ID NO: 13), it is desirable to select the 3′-region from a region downstream of the fusion point of this variant, i.e., 4129 nt (which is indicated based on SEQ ID NO: 1; the position of the fusion point is indicated hereinafter in the same manner). Specifically, the 5′-region is preferably selected from a region within 1 nt to 4079 nt of ALK gene (SEQ ID NO: 1), and the 3′-region is preferably selected from a region within 4129 nt to 6222 nt thereof. Furthermore, there is a possibility that unknown ALK fusion genes in which more bases in the 5′-end region of exon 20 are lost may be present. Therefore, in view of detecting ALK fusion genes comprehensively, the 3′-region is preferably a region at a sufficient distance, e.g., about 200 bp or more, preferably about 500 bp or more, from the fusion point of 4129 nt, which is the known fusion point located nearest to the 3′-end. It is also preferred that the 3′-region be selected from the kinase domain of ALK gene. For instance, in the Examples described below, the expression of a region of 2486 nt to 2629 nt of SEQ ID NO: 1 is measured as the 5′-region, and the expression of a region of 4801 nt to 4865 nt or 4775 nt to 4939 nt of SEQ ID NO: 1 is measured as the 3′-region. However, the present invention is not restricted to such a specific example.

The sample used in the present invention is a protein sample or a nucleic acid sample separated from a living body, preferably a nucleic acid sample. In particular, an RNA sample including a total RNA sample extracted from a sample such as cells, tissues, blood or the like collected from a living body is preferred. An RNA sample may be used in the form of cDNA reverse transcribed therefrom. RNA extraction and cDNA synthesis per se are well-known conventional methods, and may be easily carried out using commercially available kits. Random hexamers are preferably used as a primer for reverse transcription reaction.

Although measurement of expressions of the 5′-region and the 3′-region may be carried out on a protein sample by immunoassay using antibodies which specifically recognize the 5′-region and the 3′-region, respectively, in the present invention the measurement is preferably carried out by measuring the 5′-region and the 3′-region of mRNA transcribed from the target gene or cDNA synthesized from mRNA. Examples of the method for measurement of mRNA or cDNA include a nucleic acid amplification method in which primers are used or a hybridization method in which probes are used. Examples of the nucleic acid amplification method include RT-PCR, real-time PCR, NASBA and the like. Examples of the hybridization method include Northern blotting, in situ hybridization, array analysis in which immobilized probes are used, and the like. These methods per se are well-known conventional methods, and any of such methods may be used in the present invention. Immunoassay per se, and a method for producing an antibody which specifically recognizes a desired region are also well-known conventional methods.

As primers and probes, polynucleotides which specifically hybridize with the 5′-region and the 3′-region to be measured, respectively, are used. The term “specifically hybridize” used herein means that a certain polynucleotide hybridizes only with the mRNA or cDNA to be measured and does not substantially hybridize with the other nucleic acids under ordinary hybridization conditions. The term “ordinary hybridization condition” refers to a condition used for annealing in the ordinary PCR or the ordinary detection with probes. For example, in the case of PCR with Taq polymerase, the term refers to a reaction condition at an appropriate annealing temperature of about 54° C. to 60° C. using a common buffer such as one containing 50 mM KCl, 10 mM Tris-HCl (pH 8.3 to 9.0) and 1.5 mM MgCl₂. In the case of Northern hybridization, the term refers to a reaction condition at an appropriate hybridization temperature of 42° C. to 65° C. using a common hybridization solution such as one containing 5×SSPE, 50% formamide, 5×Denhardt's solution and 0.1 to 0.5% SDS. It should be noted, however, that the appropriate annealing temperature and hybridization temperature are not restricted to those exemplified above, and may be determined based on the Tm of the polynucleotide used as a primer or a probe and on the empirical rules. Those skilled in the art can easily determine the appropriate temperature. The term “does not substantially hybridize” means that a hybridization does not occur at all or, even if it occurs, the degree of the hybridization with regions other than the target region is drastically lower than that of the hybridization with the target region so that the hybridization with other regions can be relatively ignored. Those skilled in the art can appropriately design and prepare such polynucleotides, referring to known sequence information. The sequence information can be easily obtained from databases such as GenBank. For example, the base sequence of ALK gene has been registered under the accession No. NM_{—}004304 in GenBank, which sequence is also shown in SEQ ID NO: 1 of the SEQUENCE LISTING of the present application.

Specific examples of the measurement method by RT-PCR or real-time PCR include those described in the following Examples. More particularly, cDNA is prepared from each RNA sample extracted from various cells using random hexamers, and then PCR is carried out using the prepared cDNA as a template and using primer sets which specifically amplify the 5′-region and the 3′-region, respectively, of the target gene such as ALK gene, so that expressions of the respective regions of the target gene can be measured. As for real-time PCR, detection technique using fluorescently-labeled probe is also known, by which a step of electrophoresis of amplification products can be omitted so that detection can be carry out more simply and more rapidly. In the present invention, fluorescently-labeled probes which specifically hybridize with the fragments amplified from the respective regions may be prepared and used. The amplified fragments can be measured distinctively when the 5′-region and the 3′-region are respectively labeled with fluorescent labels whose wavelengths are different from each other. Those skilled in the art can easily prepare such fluorescently-labeled probes, and a specific example of the probe preparation is described in the following Examples. The term “specifically amplify” as used herein means that only the target region is amplified, or although any regions other than the target region are also amplified, the amount of the amplification thereof is drastically lower than that of the amplification of the target region so that the amplification of other regions can be relatively ignored. A set of primers which specifically hybridize with a region within the 5′-region is a primer set which specifically amplifies the 5′-region. The amplification size may be appropriately selected by those skilled in the art, and is usually not more than 1000 bp, preferably about 30 bp to 500 bp, from the viewpoint of shortening the amplification time, convenience of electrophoretic separation, and so on. For example, in the case where the target gene is ALK gene, specific examples of the primer set which specifically amplifies the 5′-region include, but not limited to, the primer set shown in SEQ ID NOs: 3 and 4; and specific examples of the primer set which specifically amplifies the 3′-region include, but not limited to, the primer set shown in SEQ ID NOs: 5 and 6, and the primer set shown in SEQ ID NOs: 20 and 21. Specific examples of the fluorescently-labeled probe include, but not limited to, the ALK 5′-region specific probe composed of the base sequence shown in SEQ ID NO: 19, which can be used in combination with the primer set shown in SEQ ID NOs: 3 and 4; and the ALK 3′-region specific probe composed of the base sequence shown in SEQ ID NO: 22, which can be used in combination with the primer set shown in SEQ ID NOs: 20 and 21.

In the nucleic acid amplification method, one or a plurality of internal control genes may be measured. As an internal control gene, GAPDH gene, ACTB gene, HPRT1 gene, HMBS gene, TBP gene and the like are well known, and these genes may also be used in the present invention. Fluorescently-labeled probes may also be prepared and used for internal control genes. In the following Examples, TBP gene is used as an internal control, and the base sequences of the primer set for TBP gene are shown in SEQ ID NOs: 7 and 8 or SEQ ID NOs: 23 and 24, but not limited thereto. Specific examples of the TBP gene-specific probe include a probe composed of the base sequence shown in SEQ ID NO: 25, which can be used in combination with the primer set shown in SEQ ID NOs: 23 and 24, but not limited thereto.

After measuring expressions of the 5′-region and the 3′-region, the expressions of the respective regions are compared with each other. In the case where the measurement is carried out by real-time PCR, the expression levels of the regions of the target gene may be normalized to the expression level of the internal control gene and then compared with each other. In the case where amplification products are electrophoresed, the presence or signal intensities of bands may be compared with each other. If the full-length target gene is expressed in the body tissue or cells from which the sample derived, the expressions of both the 5′-region and the 3′-region are observed. If only the fusion gene(s) is(are) expressed therein, the expression of the 5′-region is not observed, and only the expression of the 3′-region is observed. If neither the full length target gene nor the fusion gene(s) is(are) expressed therein, neither the expression of the 5′-region nor the expression of the 3′-region is observed. If both the full length target gene and the fusion gene(s) are expressed, the expressions of both the 5′-region and the 3′-region are observed, and in addition, the expression level of the 3′-region is higher when compared to the case where only the full length target gene is expressed. In such a case, the expression of the fusion gene(s) may be confirmed in more detail by, for example, concurrently carrying out the measurement of a control sample prepared from a tissue or cells in which the full length target gene is expressed but no fusion gene is expressed; determining the ratio of the expression levels of the 5′-region and the 3′-region in the control sample; and then comparing the ratio of the expression levels in the test sample with the ratio in the control sample. Those skilled in the art may easily prepare such control samples referring to known information. For example, in the case of ALK gene, examples of the known cell lines in which full length ALK gene is expressed and none of the ALK fusion genes is expressed include SK-N-DZ cells and the like. Thus, the expression of the fusion gene(s) can be measured based on whether the 5′-region and the 3′-region of the target gene are expressed or not or based on the ratio of the expression levels of the two regions.

For example, it is known that ALK fusion gene is expressed in lung cancer cells of some non-small-cell lung cancer patients although the full length ALK gene is not expressed in human lung. Therefore, when the present invention is carried out on RNA samples extracted from lung biopsies obtained from lung cancer patients, the expression of the fusion gene(s) can be detected based on whether the 5′-region is expressed or not. That is, in this case, if the expression of the 3′-region is observed and the expression of the 5′-region is not observed in a certain patient, then it can be determined that the ALK fusion gene(s) is(are) present and expressed in the patient. The ALK fusion gene(s) can also be detected based on whether the 5′-region and the 3′-region are expressed or not or based on the ratio of the expressions thereof when the invention is carried out on RNA samples extracted from blood samples (whole blood, serum, or plasma). ALK inhibitors are effective for treatment of cancer in lung cancer patients with ALK fusion gene expression, and the method of the present invention makes it possible to select patients who should be treated with ALK inhibitors. ALK fusion genes are known to be involved in not only lung cancer but also other various cancers, and ALK inhibitors are also effective for treatment of various cancers besides lung cancer. Therefore, the method of the present invention is also useful for cancers as well as lung cancer. Moreover, in addition to ALK fusion genes, there are other genes known to be involved in cancers, and it is known that inhibitors are also effective for treatment of such cancers. Therefore, the method of the present invention is also useful for cancers in which fusion genes other than ALK fusion genes are involved.

The two primer sets composed of the primer set for amplification of the ALK 5′-region (SEQ ID NOs: 3, 4) and the primer set for amplification of the ALK 3′-region (SEQ ID NOs: 5, 6 or SEQ ID NOs: 20, 21) used in the Examples described below are especially useful as a reagent for detection of an ALK fusion gene(s). The reagent may consist only of primers, or may contain various additives useful for stabilization of primers and the like. The reagent may further contain a primer set(s) for internal control. Moreover, the reagent may further contain a fluorescently-labeled probe(s) for real-time PCR. Requirements for the primer set and the fluorescently-labeled probe are as described above. In particular, a combination of the two primer sets described above and the primer set for internal control shown in SEQ ID NOs: 7, 8 or SEQ ID NOs: 23, 24 is a preferred combination established by the present inventor as a combination which can attain simultaneous amplification while reducing non-specific amplification through examination of more than 50 kinds of primer sets. This combination of the three sets makes it especially simple to detect the presence of the ALK fusion gene(s) (see, Examples below).

### EXAMPLES

The present invention will now be described more concretely by way of an example thereof. However, the present invention is not restricted to the Examples below.

### Example 1

### Preparation and Analysis of RNA Samples 1

H2228 cells (human lung cancer-derived cell line, ATCC), A549 cells (human lung cancer-derived cell line, ATCC), and SK-N-DZ cells (human neuroblastoma-derived cell line, ECACC) were seeded in a culture vessel of 6-well culture plate at a cell density of 20000 cells/cm², and cultured for 2 days to obtain samples. RNA extraction from the samples was carried out using a commercially available RNA extraction kit (Rneasy mini kit, Quiagen) in accordance with the manual to obtain RNA samples. The extracted RNA samples were reverse transcribed into DNA using RT primer (random hexamers, Invitrogen) and Super Script II reverse transcriptase (produced by Invitrogen), thereby obtaining cDNA samples.

The 5′-region and the 3′-region of ALK gene and TBP gene as an internal control gene which were contained in the cDNA samples were measured by real-time PCR using SYBR Green Realtime PCR Master Mix—Plus—(produced by TOYOBO). The primers used are shown below. The reaction conditions were as follows: thermal denaturation at 96° C. for 120 sec, followed by cycle reaction of 96° C. for 10 sec, annealing at 60° C. for 30 sec, extension at 72° C. for 30 sec; the cycle number was appropriately determined by monitoring real-time PCR. The primers used are shown below. As for the composition of the reaction solution, each primer was used at a concentration of 0.8 μM, and buffer etc. attached to the kit were used in accordance with the attached instructions.

### ALK 5′-Region:

forward (cccgcttctgaaagtgctac, SEQ ID NO: 3), reverse (cccggttttgttctccacta, SEQ ID NO: 4) ALK 3′-Region:
forward (agaggccttcatggaaggaa, SEQ ID NO: 5), reverse (atagcagcactccaaaggac, SEQ ID NO: 6)

### TBP Gene:

forward (ccaaggaattgaggaagttgc, SEQ ID NO: 7), reverse (gtgccataaggcatcattgg, SEQ ID NO: 8)

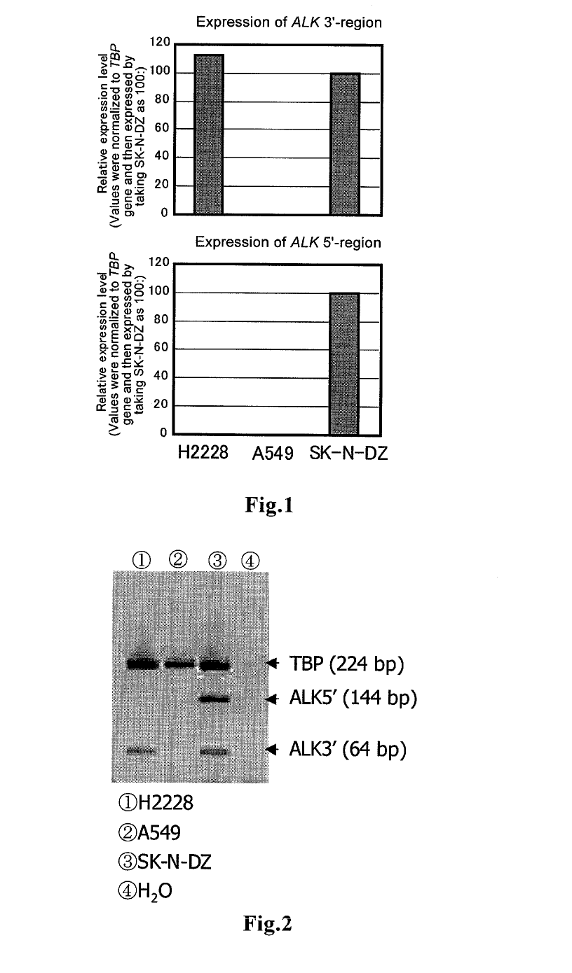

The results are shown in FIG. 1. The expression levels of ALK gene normalized to the TBP gene expression level were expressed as relative values taking the expression levels in SK-N-DZ cells as 100%. The presence of the ALK5′-region and the ALK3′-region was confirmed in the cDNA sample prepared from SK-N-DZ cells, indicating that only the normal, unfused ALK gene was present. In the sample prepared from H2228 cells, only the ALK 3′-region was confirmed to be present, indicating that the ALK 5′-region was not expressed and that only the fused ALK 3′-region was expressed. These results indicate that the presence of the EML4-ALK fusion gene(s) can be detected simply by measuring the presence of the 5′-region and the 3′-region of ALK gene.

### Example 2

### Preparation and Analysis of RNA Samples 2

cDNA samples were prepared from each cells in the same manner as in Example 1. The 5′-region and the 3′-region of ALK gene and TBP gene as an internal control gene which were contained in the cDNA samples were amplified by multiplex PCR using TITANIUM Taq DNA polymerase (produced by Takara Bio Inc.), and electrophoresis was carried out. The same primers as those used in Example 1 were used. PCR conditions were as follows; thermal denaturation at 96° C. for 120 sec, followed by 35 cycles of 96° C. for 15 sec and 68° C. for 60 sec. As for the composition of the reaction solution, each primer was used at a concentration of 0.8 μM, and buffer etc. attached to the kit were used in accordance with the attached instructions.

The results are shown in FIG. 2. The amplification of the ALK 5′-region and the ALK 3′-region was confirmed in the cDNA sample prepared from SK-N-DZ cells, whereas, the amplification of only the ALK 3′-region was confirmed in H2228 cells. The amplification of the internal control TBP gene was confirmed in all the cDNA samples. These results indicate that the presence of the EML4-ALK fusion gene(s) can be detected simply by measuring the presence of the 5′-region and the 3′-region of ALK gene and an internal control gene.

### Example 3

RNA samples were obtained from each cells in the same manner in Example 1. The 5′-region and the 3′-region of ALK gene and TBP gene as an internal control gene which were contained in the RNA samples were measured by One-Step real-time PCR using TITANIUM One-Step RT-PCR Kit (produced by Takara Bio Inc.). The below-described primers and probes for specific detection of the respective amplification products were used. The probes were labeled at the 3′-end with any one of three fluorescent substances whose wavelength was different from one another, which makes it possible to measure the amount of the amplification products by fluorescence quenching caused by a guanine base as known in the art. The reaction conditions were as follows: 50° C. for 1 hour, and thereafter thermal denaturation at 96° C. for 120 sec, followed by cycle reaction of 96° C. for 10 sec, annealing at 60° C. for 30 sec, extension at 72° C. for 30 sec; the cycle number was appropriately determined by monitoring real-time PCR. As for the composition of the reaction solution, each primer was used at a concentration of 0.8 each probe was used at a concentration of 0.1 μM, and buffer etc. attached to the kit were used in accordance with the attached instructions.

ALK 5′-region:
forward (cccgcttctgaaagtgctac, SEQ ID NO: 3),
reverse (cccggttttgttctccacta, SEQ ID NO: 4),
probe (tctccatgtgagctccgaatgtcc, the cytosine
base at 3′-end was labeled with TAMRA, SEQ
ID NO: 19)
ALK 3′-region:
forward (atgctgccagttaagtggatg, SEQ ID NO: 20),
reverse (actggtgacaaactccagaac, SEQ ID NO: 21),
probe (tatgccataccccagcaaaagcaac, the cytosine
base at 3′-end was labeled with BODIPY FL,
SEQ ID NO: 22)
TBP gene:
forward (cttggcgtgtgaagataacc, SEQ ID NO: 23),
reverse (tgctgcctttgttgctcttc, SEQ ID NO: 24),
probe (ccttacgctcagggcttggcctcc, the cytosine
base at 3′-end was labeled with 5-CR6G, SEQ
ID NO: 25)

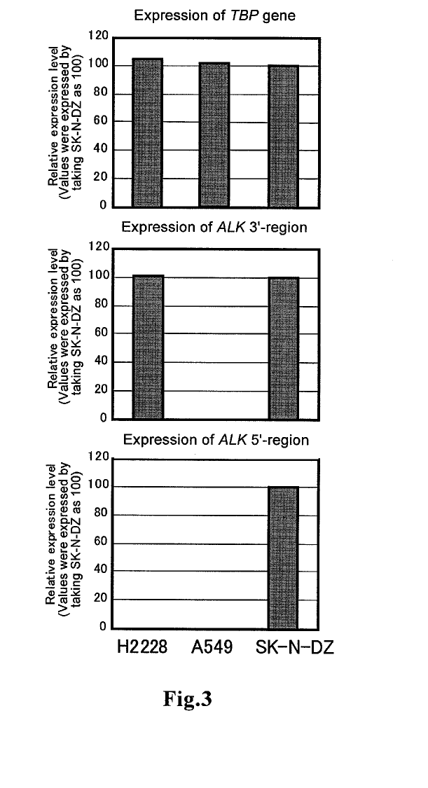

The results are shown in FIG. 3. The expression levels of TBP gene, ALK 5′-region and ALK 3′-region were expressed as relative values taking the expression levels in SK-N-DZ cells as 100%. The amplification of the ALK 5′-region and the ALK 3′-region was observed in RNA sample derived from SK-N-DZ cells, whereas, the amplification of only the ALK 3′-region was observed in H2228 cells. The amplification of internal control TBP gene was observed in all the RNA samples. These results indicate that the presence of the EML4-ALK fusion gene(s) can be detected directly by measuring the presence of the 5′-region and the 3′-region of ALK gene and an internal control gene.

1. A method for measuring a fusion gene(s), which is applied to a sample separated from a living body, said method comprising: measuring expressions of a 5′-region and a 3′-region of one of the component genes of the fusion gene(s) which may be present in the living body, wherein said 5′-region is upstream of and said 3′-region is downstream of a fusion point in said one of the component genes; and comparing the expression of the 5′-region with the expression of the 3′-region.

2. The method according to claim 1, wherein said one of the component genes is ALK gene.

3. The method according to claim 2, wherein said 5′-region is any region within the region of exons 1 to 19 of ALK gene, and said 3′-region is any region within the region of exons 20 to 29.

4. The method according to claim 3, wherein said 5′-region is any region within the 1 nt to 4079 nt region of the ALK gene sequence shown in SEQ ID NO: 1, and said 3′-region is any region within the 4129 nt to 6222 nt region of said ALK gene sequence.

5. The method according to any one of claims 1 to 4, wherein said sample is an RNA sample, and the expression measurement is carried out by measuring the 5′-region and the 3′-region of mRNA transcribed from ALK gene or cDNA synthesized from said mRNA.

6. The method according to claim 5, which method is carried out by RT-PCR using a primer set which specifically amplifies the 5′-region and a primer set which specifically amplifies the 3′-region.

7. The method according to claim 6, wherein a 5′-region detection primer set composed of the base sequences shown in SEQ ID NOs: 3 and 4 of the SEQUENCE LISTING, respectively, and a 3′-region detection primer set composed of the base sequences shown in SEQ ID NOs: 5 and 6, respectively, are used.

8. The method according to claim 7, wherein an internal control primer set composed of the base sequences shown in SEQ ID NOs: 7 and 8, respectively, is further used.

9. The method according to claim 2, wherein said living body is a cancer patient.

10. The method according to claim 9, wherein said living body is a lung cancer patient.

11. A reagent for detection of a fusion gene(s) foimed by fusion with ALK gene, said reagent comprising a 5′-region detection primer set composed of the base sequences shown in SEQ ID NOs: 3 and 4 of the SEQUENCE LISTING, respectively, and a 3′-region detection primer set composed of the base sequences shown in SEQ ID NOs: 5 and 6, respectively.

12. A reagent for detection of a fusion gene(s) formed by fusion with ALK gene, said reagent comprising a 5′-region detection primer set composed of the base sequences shown in SEQ ID NOs: 3 and 4 of the SEQUENCE LISTING, respectively, and a 3′-region detection primer set composed of the base sequences shown in SEQ ID NOs: 20 and 21, respectively.

13. The reagent according to claim 11, further comprising an internal control primer set composed of the base sequences shown in SEQ ID NOs: 7 and 8, respectively.

14. The reagent according to claim 12, further comprising an internal control primer set composed of the base sequences shown in SEQ ID NOs: 23 and 24, respectively.


SEQUENCE LISTING (25 sequences)

SEQ ID NO: 1 - DNA, 6222 bp, Homo sapiens
  CDS (908)..(5770)
gggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 2 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 3 - DNA, 20 bp, Artificial Sequence
  primer ALK_F_2486, for amplification of ALK 5′ region
cccgcttctgaaagtgctac

SEQ ID NO: 4 - DNA, 20 bp, Artificial Sequence
  primer ALK_R_2629, for amplification of ALK 5′ region
cccggttttgttctccacta

SEQ ID NO: 5 - DNA, 20 bp, Artificial Sequence
  primer ALK_F_4801, for amplification of ALK 3′ region
agaggccttcatggaaggaa

SEQ ID NO: 6 - DNA, 20 bp, Artificial Sequence
  primer ALK_R_4865, for amplification of ALK 3′ region
atagcagcactccaaaggac

SEQ ID NO: 7 - DNA, 21 bp, Artificial Sequence
  primer TBP_F_115, for measurement of housekeeping gene TBP
ccaaggaattgaggaagttgc

SEQ ID NO: 8 - DNA, 20 bp, Artificial Sequence
  primer TBP_R_339, for measurement of housekeeping gene TBP
gtgccataaggcatcattgg

SEQ ID NO: 9 - DNA, 3926 bp, Homo sapiens
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 10 - DNA, 4679 bp, Homo sapiens
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtacttgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 11 - DNA, 2473 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 12 - DNA, 2506 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagcaaaaatgtcaactcgcgaaaaaaacagccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 13 - DNA, 3409 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagatatgctggatgagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaagaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 14 - DNA, 2014 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 15 - DNA, 2131 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggttcagagctcaggggaggatatggagatccagggaggcttcctgtaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 16 - DNA, 3365 bp, Homo sapiens
tactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 17 - DNA, 3435 bp, Homo sapiens
tactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgga

SEQ ID NO: 18 - DNA, 4479 bp, Homo sapiens
tgcgagaaagatggcggacctggccgagtgcaacatcaaagtgatgtgtcgcttcagacctctcaacgagtctgaagtgaaccgcggcgacaagtacatcgccaagtttcagggagaagacacggtcgtgatcgcgtccaagccttatgcatttgatcgggtgttccagtcaagcacatctcaagagcaagtgtataatgactgtgcaaagaagattgttaaagatgtacttgaaggatataatggaacaatatttgcatatggacaaacatcctctgggaagacacacacaatggagggtaaacttcatgatccagaaggcatgggaattattccaagaatagtgcaagatatttttaattatatttactccatggatgaaaatttggaatttcatattaaggtttcatattttgaaatatatttggataagataagggacctgttagatgtttcaaagaccaacctttcagttcatgaagacaaaaaccgagttccctatgtaaaggggtgcacagagcgttttgtatgtagtccagatgaagttatggataccatagatgaaggaaaatccaacagacatgtagcagttacaaatatgaatgaacatagctctaggagtcacagtatatttcttattaatgtcaaacaagagaacacacaaacggaacaaaagctgagtggaaaactttatctggttgatttagctggtagtgaaaaggttagtaaaactggagctgaaggtgctgtgctggatgaagctaaaaacatcaacaagtcactttctgctcttggaaatgttatttctgctttggctgagggtagtacatatgttccatatcgagatagtaaaatgacaagaatccttcaagattcattaggtggcaactgtagaaccactattgtaatttgctgctctccatcatcatacaatgagtctgaaacaaaatctacactcttatttggccaaagggccaaaacaattaagaacacagtttgtgtcaatgtggagttaactgcagaacagtggaaaaagaagtatgaaaaagaaaaagaaaaaaataagatcctgcggaacactattcagtggcttgaaaatgagctcaacagatggcgtaatggggagacggtgcctattgatgaacagtttgacaaagagaaagccaacttggaagctttcacagtggataaagatattactcttaccaatgataaaccagcaaccgcaattggagttataggaaattttactgatgctgaaagaagaaagtgtgaagaagaaattgctaaattatacaaacagcttgatgacaaggatgaagaaattaaccagcaaagtcaactggtagagaaactgaagacgcaaatgttggatcaggaggagcttttggcatctaccagaagggatcaagacaatatgcaagctgagctgaatcgccttcaagcagaaaatgatgcctctaaagaagaagtgaaagaagttttacaggccctagaagaacttgctgtcaattatgatcagaagtctcaggaagttgaagacaaaactaaggaatatgaattgcttagtgatgaattgaatcagaaatcggcaactttagcgagtatagatgctgagcttcagaaacttaaggaaatgaccaaccaccagaaaaaacgagcagctgagatgatggcatctttactaaaagaccttgcagaaataggaattgctgtgggaaataatgatgtaaagcagcctgagggaactggcatgatagatgaagagttcactgttgcaagactctacattagcaaaatgaagtcagaagtaaaaaccatggtgaaacgttgcaagcagttagaaagcacacaaactgagagcaacaaaaaaatggaagaaaatgaaaaggagttagcagcatgtcagcttcgtatctctcaacatgaagccaaaatcaagtcattgactgaataccttcaaaatgtggaacaaaagaaaagacagttggaggaatctgtcgatgccctcagtgaagaactagtccagcttcgagcacaagagaaagtccatgaaatggaaaaggagcacttaaataaggttcagactgcaaatgaagttaagcaagctgttgaacagcagatccagagccatagagaaactcatcaaaaacagatcagtagtttgagagatgaagtagaagcaaaagcaaaacttattactgatcttcaagaccaaaaccagaaaatgatgttagagcaggaacgtctaagagtagaacatgagaagttgaaagccacagatcaggaaaagagcagaaaactacatgaacttacggttatgcaagatagacgagaacaagcaagacaagacttgaagggtttggaagagacagtggcaaaagaacttcagactttacacaacctgcgcaaactctttgttcaggacctggctacaagagttaaaaagagtgctgagattgattctgatgacaccggaggcagcgctgctcagaagcaaaaaatctcctttcttgaaaataatcttgaacagctcactaaagtgcacaaacagttggtacgtgataatgcagatctccgctgtgaacttcctaagttggaaaagcgacttcgagctacagctgagagagtgaaagctttggaatcagcactgaaagaagctaaagaaaatgcatctcgtgatcgcaaacgctatcagcaagaagtagatcgcataaaggaagcagtcaggtcaaagaatatggccagaagagggcattctgcacagattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactca

SEQ ID NO: 19 - DNA, 24 bp, Artificial Sequence
  ALK 5′ specific probe
tctccatgtgagctccgaatgtcc

SEQ ID NO: 20 - DNA, 21 bp, Artificial Sequence
  primer for detection of ALK 3′ region
atgctgccagttaagtggatg

SEQ ID NO: 21 - DNA, 21 bp, Artificial Sequence
  primer for detection of ALK 3′ region
actggtgacaaactccagaac

SEQ ID NO: 22 - DNA, 25 bp, Artificial Sequence
  ALK 3′ specific probe
tatgccataccccagcaaaagcaac

SEQ ID NO: 23 - DNA, 20 bp, Artificial Sequence
  primer for measurement of housekeeping gene TBP
cttggcgtgtgaagataacc

SEQ ID NO: 24 - DNA, 20 bp, Artificial Sequence
  primer for measurement of housekeeping gene TBP
tgctgcctttgttgctcttc

SEQ ID NO: 25 - DNA, 24 bp, Artificial Sequence
  TBP gene specific probe
ccttacgctcagggcttggcctcc